(19) United States
(12) Patent Application Publication
(10) Pub. No.: US 2021/0386380 A1
(43) Pub. Date: Dec. 16, 2021

(54) SYSTEMS AND METHODS FOR COLLECTING RETINAL SIGNAL DATA AND REMOVING ARTIFACTS

(71) Applicant: DIAMENTIS INC., Quebec (CA)

(72) Inventor: Claude HARITON, Saint Antoine de Tilly (CA)

(21) Appl. No.: 17/345,419

(22) Filed: Jun. 11, 2021

Related U.S. Application Data
(60) Provisional application No. 63/038,257, filed on Jun. 12, 2020.
(60) Provisional application No. 63/149,508, filed on Feb. 15, 2021.

(30) Foreign Application Priority Data
Mar. 25, 2021 (CA) PCT/CA2021/050390

(51) Int. Cl. A61B 5/00; A61B 5/398

(52) U.S. Cl. CPC A61B 5/7207; A61B 2560/0223; A61B 5/398

(57) ABSTRACT

There is disclosed a method and system for generating retinal signal data. Calibration data corresponding to an individual may be received. A threshold impedance may be determined based on the calibration data. Retinal signal data corresponding to the individual may be received. The impedance of the circuit collecting the retinal signal data may be compared to the threshold impedance to determine whether the retinal signal data contains any artifacts. A portion of the retinal signal data corresponding to the artifacts may be removed from the retinal signal data.

### CROSS-REFERENCE TO RELATED APPLICATIONS

This application claims the benefit of U.S. Provisional Patent Application No. 63/038,257, filed Jun. 12, 2020, U.S. Provisional Patent Application No. 63/149,508, filed Feb. 15, 2021, International Application No. PCT/CA2021/050390 and U.S. patent application Ser. No. 17/212,410, both filed on Mar. 25, 2021. Each of the applications named in this paragraph are incorporated by reference herein in their entirety.

### FIELD

The present technology relates to systems and methods for collecting and/or processing retinal signal data generated by light stimulation.

### BACKGROUND

A signal is a function that conveys information generally about the behavior of a physical or physiological system, or the attributes of some phenomenon. Signal processing is the process of extracting information from a signal. Retinal signal data, such as electroretinograms (ERG) data, may be collected for analysis. The retinal signal data may be collected using sensors such as one or more electrodes attached to an individual. The electrodes may capture electrical signals. A light stimulator may be used to trigger the electrical signals. The retinal signal data may be used by a medical practitioner as a diagnostic aid.

During the capture of retinal signal data, an individual's movements may affect the retinal signal data. This may be more common for individuals that are subject to mental conditions, as these individuals may find it more difficult to remain still while the retinal signal data is captured. Also, these movements may be more likely to occur when the amount of time that the retinal signal data is recorded is extended. It is an object of the present technology to ameliorate at least some of the limitations present in the prior art.

### SUMMARY

Embodiments of the present technology have been developed based on developers' appreciation of certain shortcomings associated with existing systems collecting, processing, and/or analyzing retinal signal data. The retinal signal data may include artifacts. These artifacts may impede further analysis of the retinal signal data. It may be preferable to use retinal signal data that does not contain artifacts and/or that contains less artifacts. A dynamic resistance of a circuit collecting the retinal signal data, such as the impedance of the circuit, may be used to determine whether the retinal signal data contains artifacts.

Embodiments of the present technology have been developed based on the developers' observation that data obtained in electroretinograms (ERG) may provide some insight into determining conditions, such as medical conditions. However, existing methods to collect and analyse electroretinograms (ERG) can only collect and analyse a limited volume of information from the captured electrical signals. It was found that expansion of the volume of information collected regarding retinal response to light stimulation allowed generating retinal signal data with a higher density of information, a higher volume of information, and/or additional types of information. This retinal signal data enables a multimodal mapping of the electrical signals and/or other data and allows the detection of additional features in the multimodal mapping specific to certain conditions. The multimodal mapping may include multiple parameters of the retinal signal data, such as time, frequency, light stimulation parameters, and/or any other parameter.

Several parameters or data which have a direct impact on the electrical signals might not be collected during conventional ERG recording. However, the triggered electrical signals may be directly dependent on those parameters. These parameters can include real-time measurement of light spectrum, light intensity, illuminated area, and/or impedance of the circuit collecting the electrical signals.

Embodiments of the present technology form the basis for collecting and/or processing of retinal signal data which has more volume of information, more density of information and/or additional types of information detail compared to conventional ERG data. The number and/or range of light intensities of the light stimulation may be increased. This retinal signal data allows, in certain embodiments, the mathematical modeling of datasets containing a multiplicity of information, identification of retinal signal features, and the ability to identify biomarkers and/or biosignatures in the retinal signal data using for example the retinal signal features. Certain, non-essential, embodiments of the present technology also provide methods for collecting the retinal signal data which has more volume of information, more density of information and/or additional types of information compared to conventional ERG data.

In some instances, the retinal signal data, or any other signal data associated with light stimulation may contain artifacts. The artifacts may include distorted signals, interferences, and/or any other type of artifacts. The artifacts may occur through one or more of: signals not originating from the retina being inadvertently captured, shifts in the electrode positioning, changes in the ground or reference electrode contact, photomyoclonic reflex, eye lid blinks, ocular movements, and/or external electrical interferences. These artifacts may restrain further analysis of the retinal signal data, or skew the further analysis. It would be beneficial if these artifacts could be removed, compensated for, or prevented.

Parameters of the electrical signals emitted by an individual may be measured, such as voltage, current, impedance, and/or any other parameters. The parameters may be measured continuously over a period of time. During the period of time, the individual may be exposed to a flash of light. The data collected prior to the flash of light may be used as calibration data. The data collected after the flash of light may be retinal signal data. Baseline parameters of the electrical circuit capturing the electrical signals may be determined using the calibration data, such as a baseline voltage, baseline current, baseline impedance, and/or any other parameters. A threshold impedance may be determined based on the baseline impedance. The retinal signal data may be compared to the threshold impedance. If the impedance of the circuit during collection of the retinal signal data surpasses the threshold impedance, the retinal signal data may be determined to have artifacts. An amount of change of the impedance of the circuit and/or a rate of change of the impedance may also be determined to indicate a presence of an artifact.

In conventional ERG, a flash of light having the same parameters may be repeated multiple times, such as ten times. The electrical signals responsive to the flash may be collected each time. Data regarding those electrical signals may be averaged, such as by determining an average voltage of the electrical signals. The same flash of light (i.e. a flash of light having the same flash parameters) may be repeated to reduce the impact of artifacts on the collected data. For example if the flash of light is repeated ten times, and artifacts occur in the electrical signals responsive to one of those flashes, the impact of those artifacts will be reduced by combining the data collected after that flash of light with the data collected after the other nine flashes of light.

Artifacts may be detected through other means, such as by monitoring the dynamic resistance of the collecting circuit, such as the impedance, admittance, and/or susceptance of the circuit collecting the electrical signals. Rather than repeating the same flash of light multiple times, retinal signal data responsive to a single flash of light and/or a reduced number of flashes of light may be collected. The retinal signal data may be analyzed to determine whether the retinal signal data contains artifacts. For example the impedance of the retinal signal data may be compared to a threshold impedance. If the impedance of the retinal signal data does not exceed the threshold impedance, the retinal signal data may be determined not to contain artifacts. The retinal signal data may then be stored. In this manner, retinal signal data may be collected without repeating the flash of light having the same parameters and/or the amount of times that a flash of light having the same parameters is repeated may be reduced. This may reduce the amount of time used for collecting the retinal signal data and/or decrease the impact of artifacts on the retinal signal data.

In certain embodiments, a more efficient processing of retinal signal data is possible compared to ERG data. The advantage of retinal signal data as compared to the conventional ERG data, is to benefit from a larger amount of information related to the electrical signals and additional retinal signal features. This additional data may be used to identify artifacts in the retinal signal data, remove the artifacts in the retinal signal data, reduce the artifacts in the retinal signal data, and/or otherwise compensate for the artifacts in the retinal signal data.

In certain embodiments, artifacts are detected and/or removed from the retinal signal data. The artifacts may be detected and/or removed after the collection of retinal signal data is complete and/or in real-time during the collection of the retinal signal data. If the artifacts are detected during collection of the retinal signal data, an indication may be displayed to an operator that artifacts have been detected. The parameters of the flash of light that was triggered prior to the retinal signal data with artifacts may be determined and a flash of light having the same parameters may be triggered. Retinal signal data occurring after that flash of light may be captured and/or stored for further analysis.

According to a first broad aspect of the present technology, there is provided a method executed by at least one processor of a computing system, the method comprising: receiving retinal signal data corresponding to an individual; determining that there are one or more artifacts in the retinal signal data by determining that an impedance of a circuit that collected the retinal signal data has surpassed a threshold impedance of the circuit; modifying the retinal signal data to compensate for the artifacts; and storing the retinal signal data.

In some implementations of the method, modifying the retinal signal data to compensate for the artifacts comprises removing at least a portion of the retinal signal data corresponding to the artifacts.

In some implementations of the method, the method further comprises: receiving calibration data corresponding to the individual; and determining, based on the calibration data, the threshold impedance of the circuit.

In some implementations of the method, the retinal signal data is responsive to at least one flash of light from a light stimulator, wherein the calibration data is collected prior to the at least one flash of light by the same circuit that collected the retinal signal data, and wherein the method further comprises causing the light stimulator to generate the at least one flash of light.

In some implementations of the method, the retinal signal data has a sampling frequency between 4 to 24 kHz.

In some implementations of the method, the retinal signal data is collected for a signal collection time of 200 milliseconds to 500 milliseconds.

In some implementations of the method, the one or more artifacts comprise distortions in the retinal signal data.

In some implementations of the method, the one or more artifacts were caused by one or more of: capture of electrical signals not originating from the retina, shift in electrode positioning, change in ground or reference electrode contact, photomyoclonic reflex, eye lid blinks, and ocular movements.

In some implementations of the method, the method further comprises: extracting, from the retinal signal data, one or more retinal signal features; extracting, from the retinal signal features, one or more descriptors; applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and outputting the first predicted probability and the second predicted probability.

According to another broad aspect of the present technology, there is provided a method executed by at least one processor of a computing system, the method comprising: receiving retinal signal data corresponding to an individual; determining that there are one or more artifacts in the retinal signal data by determining that an impedance of a circuit that collected the retinal signal data has surpassed a threshold impedance of the circuit; storing an indication in the retinal signal data of time periods corresponding to the one or more artifacts; and storing the retinal signal data.

In some implementations of the method, the method further comprises: receiving calibration data corresponding to the individual; and determining, based on the calibration data, the threshold impedance of the circuit.

In some implementations of the method, the method further comprises: determining the time periods corresponding to the one or more artifacts by determining the time periods that an impedance of the retinal signal data surpasses the threshold impedance.

In some implementations of the method, the retinal signal data is responsive to at least one flash of light from a light stimulator, wherein the calibration data is collected prior to the at least one flash of light, and wherein the method further comprises causing the light stimulator to generate the at least one flash of light.

In some implementations of the method, the retinal signal data has a sampling frequency between 4 to 24 kHz.

In some implementations of the method, the retinal signal data is collected for a signal collection time of 200 milliseconds to 500 milliseconds.

In some implementations of the method, the one or more artifacts comprise distortions in the retinal signal data.

In some implementations of the method, the one or more artifacts were caused by one or more of: capture of electrical signals not originating from the retina, shift in electrode positioning, change in ground or reference electrode contact, photomyoclonic reflex, eye lid blinks, and ocular movements.

In some implementations of the method, the method further comprises: extracting, from the retinal signal data, one or more retinal signal features; extracting, from the retinal signal features, one or more descriptors; applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and outputting the first predicted probability and the second predicted probability.

According to another broad aspect of the present technology, there is provided a method executed by at least one processor of a computing system, the method comprising:
recording a first set of retinal signal data corresponding to an individual;
determining that there are one or more artifacts in the first set of retinal signal data by determining that an impedance of a circuit that collected the first set of retinal signal data has surpassed a first threshold impedance of the circuit; recording a second set of retinal signal data corresponding to the individual; determining that the impedance of the circuit while recording the second set of retinal signal data has not surpassed a second threshold impedance of the circuit; and storing the second set of retinal signal data.

In some implementations of the method, the method further comprises: recording a first set of calibration data corresponding to the individual before recording the first set of retinal signal data; determining, based on the first set of calibration data, the first threshold impedance of the circuit; recording a second set of calibration data corresponding to the individual before recording the second set of retinal signal data; and determining, based on the second set of calibration data, the second threshold impedance of the circuit.

In some implementations of the method, the method further comprises: after recording the first set of calibration data, triggering a light stimulator to generate a first flash of light based on a set of flash parameters, wherein the first set of retinal signal data is responsive to the first flash of light; and after recording the second set of calibration data, triggering the light stimulator to generate a second flash of light based on the set of flash parameters, wherein the second set of retinal signal data is responsive to the second flash of light.

In some implementations of the method, the first set of retinal signal data and the second set of retinal signal data have a sampling frequency between 4 to 24 kHz.

In some implementations of the method, the first set of retinal signal data and the second set of retinal signal data are collected for a signal collection time of 200 milliseconds to 500 milliseconds.

In some implementations of the method, the method further comprises: extracting, from the second set of retinal signal data, one or more retinal signal features; extracting, from the retinal signal features, one or more descriptors; applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and outputting the first predicted probability and the second predicted probability.

According to another broad aspect of the present technology, there is provided a method executed by at least one processor of a computing system, the method comprising: receiving retinal signal data corresponding to an individual; inputting the retinal signal data to a machine learning algorithm (MLA), wherein the MLA was trained using labeled retinal signal data, and wherein each set of retinal signal data in the labeled retinal signal data comprises a label indicating whether the respective set of retinal signal data comprises any artifacts; outputting, by the MLA, adjusted retinal signal data; and storing the adjusted retinal signal data.

In some implementations of the method, the retinal signal data has a sampling frequency between 4 to 24 kHz.

In some implementations of the method, the retinal signal data is collected for a signal collection time of 200 milliseconds to 500 milliseconds.

In some implementations of the method, the MLA removes portions of the retinal signal data corresponding to artifacts.

In some implementations of the method, the MLA adds indicators to the retinal signal data that indicate which portions of the retinal signal data comprise artifacts.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a block diagram of an example computing environment in accordance with various embodiments of the present technology;

FIG. 2 is a block diagram of a retinal signal data processing system in accordance with various embodiments of the present technology;

FIG. 3 is a diagram of exemplary electrode placement for collecting retinal signal data in accordance with various embodiments of the present technology;

FIG. 4 is a flow diagram of a method for compensating for artifacts in retinal signal data in accordance with various embodiments of the present technology;

FIG. 5 is a flow diagram of a method for detecting artifacts and outputting an alert during collection of retinal signal data in accordance with various embodiments of the present technology;

FIG. 6 is a flow diagram of a method for using a machine learning algorithm (MLA) to remove artifacts from retinal signal data in accordance with various embodiments of the present technology;

FIG. 7 is a flow diagram of a method for predicting a likelihood of a medical condition in accordance with various embodiments of the present technology;

FIG. 8 illustrates three-dimensional retinal signal data generated with 45 incremental light intensities (luminance steps) from 0.4 cd·sec/m² to 794 cd·sec/m² in photopic conditions (accommodation to background light) with a sampling frequency of 16 kHz in accordance with various embodiments of the present technology;

FIG. 9 is a three-dimensional impedance of retinal signal data generated with 45 incremental light intensities (luminance) from 0.4 cd·sec/m² to 794 cd·sec/m² in photopic conditions (accommodation to background light) and impedance capture simultaneously with the amplitude of the retinal signal at a sampling frequency of 16 kHz in accordance with various embodiments of the present technology;

FIG. 10 is a four-dimensional retinal signal data (amplitude vs impedance vs stimulation light luminance vs time) generated with 45 incremental light intensities (luminance) from 0.4 cd·sec/m² to 794 cd·sec/m² in photopic conditions (accommodation to background light) and simultaneous impedance capture with a sampling frequency of 16 kHz in accordance with various embodiments of the present technology;

FIG. 11 is a four-dimensional retinal signal data (amplitude vs impedance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz in accordance with various embodiments of the present technology. Changes in impedance are found during the signal recording at luminance 9 (0.9 cd·sec/m²) and 72 (624 cd·sec/m²), with impedance higher than baseline values not exceeding 500 ohms, which indicates two distortions are present in the signal;

FIG. 12 is a four-dimensional retinal signal (current vs admittance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz in accordance with various embodiments of the present technology. The changes in impedance found during the signal recording presented in FIG. 11, respectively at luminance 9 (0.9 cd·sec/m²) and 72 (624 cd·sec/m²), have been rejected by the present technology and the signal has been corrected accordingly; and

FIG. 13 is a four-dimensional retinal signal (current vs admittance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz. The two distortions found in the retinal signal recording presented in FIG. 11, respectively at luminance 9 (0.9 cd·sec/m²) and 72 (624 cd·sec/m²), have been corrected.

It should be noted that, unless otherwise explicitly specified herein, the drawings are not to scale.

### DETAILED DESCRIPTION

Certain aspects and embodiments of the present technology are directed to methods and systems for collecting retinal signal data. Broadly, certain aspects and embodiments of the present technology comprise a process to obtain retinal signal data by e.g. enlarging the conditions for light stimulation (e.g. number and range of light intensities), recording the dynamic resistance (impedance) of the circuit used to collect the retinal signal in the electrical components of the signal itself, capturing retinal signal data for a longer period of time, and/or capturing retinal signal data at a higher frequency (sampling rate). The retinal signal data may be analysed and/or processed to remove artifacts in the retinal signal data. The artifacts may be caused by capture of electrical signals which are not originating from the retina. The artifacts may include distorted electrical signals in the retinal signal data which may have occurred due to, e.g., shift in the electrode positioning or contact with the surface from where the signal is collected, change in the ground or reference electrode contact, photomyoclonic reflex, eye lid blinks, and/or ocular movements. The artifacts may be detected and/or removed based on impedance values of the electrical circuit used to collect the retinal signal data. Signal amplitude values of the retinal signal data may be corrected based on the impedance values. Portions of the retinal signal data corresponding to the artifacts may be removed from the retinal signal data.

The characteristics of light stimulation, e.g. light spectrum, light intensity, and/or duration of the light stimulation or the surface illuminated may have a direct impact on the electrical signals that are triggered by the light stimulation. These characteristics may be measured, such as in real-time during collection of the retinal signal data. These characteristics may lead to a more accurate recording and/or analysis of the electrical signals.

Certain aspects and embodiments of the present technology provide methods and systems that can convert the retinal signal data (voltage amplitude) in electric current values (flow of electric charges) by using the real-time recording of impedance. This conversion may be performed in real-time during collection of the retinal signal data.

Certain aspects and embodiments of the present technology provide methods and systems that can detect the occurrence of artifacts by analysing the impedance of the circuit collecting the electrical signals (including some or all of the electrodes part of that circuit). The detection of artifacts may be performed in real-time during collection of the retinal signal data.

Certain aspects and embodiments of the present technology provide methods and systems that can correct artifacts by converting the retinal signal data into current and analysing the time-current function as opposed to the time-voltage function.

Certain aspects and embodiments of the present technology provide methods and systems that can remove artifacts by reconstructing the retinal signal data based upon predefined impedance thresholds.

The systems and methods described herein may be fully or at least partially automated so as to minimize an input of a clinician in collecting and/or processing the retinal signal data.

The systems and methods described herein may be based on retinal signal data having a higher level of information compared to data captured by conventional ERG. The collected retinal signal data may be analyzed using mathematical and statistical calculations to extract specific retinal signal features. The retinal signal features may comprise parameters of the retinal signal data and/or features generated using the retinal signal data. Descriptors may be extracted from the retinal signal features. Graphical representations of the findings may be developed and output, and may provide visual support for choices made in selecting relevant retinal signal features and/or descriptors. Applications may apply mathematical and/or statistical analysis of the results, allowing the quantification of those retinal signal features and/or descriptors, and comparisons between various conditions. Based upon the retinal signal data and/or any other clinical information, classifiers may be constructed which describe a biosignature of a condition identified in the retinal signal data. The retinal signal data of an individual may be collected, and a distance between the individual's retinal signal data and the identified biosignatures may be determined, such as by using the classifiers.

### Computing Environment

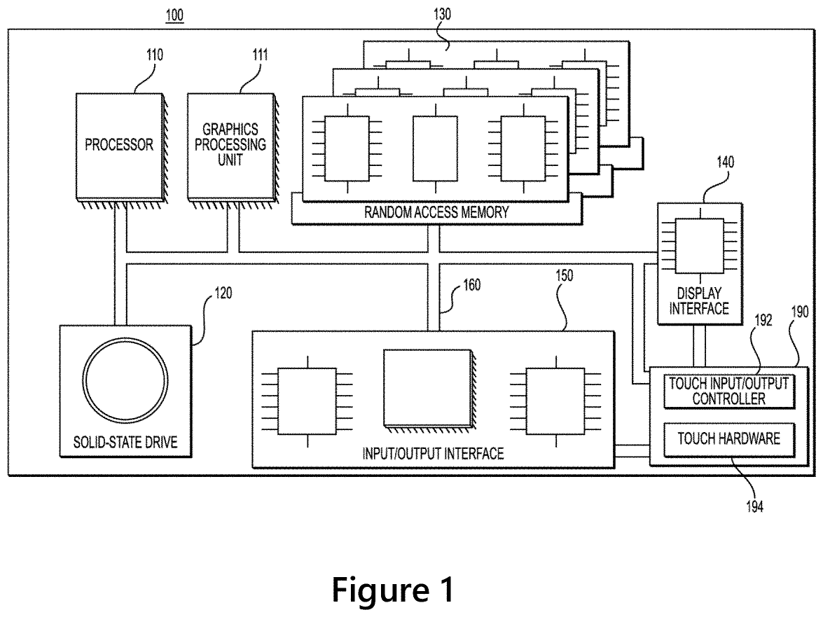

FIG. 1 illustrates a computing environment 100, which may be used to implement and/or execute any of the methods described herein. In some embodiments, the computing environment 100 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 100 comprises various hardware components including one or more single or multi-core processors collectively represented by processor 110, a solid-state drive 120, a random access memory 130, and an input/output interface 150. The computing environment 100 may be a computer specifically designed to operate a machine learning algorithm (MLA). The computing environment 100 may be a generic computer system.

In some embodiments, the computing environment 100 may also be a subsystem of one of the above-listed systems. In some other embodiments, the computing environment 100 may be an “off-the-shelf” generic computer system. In some embodiments, the computing environment 100 may also be distributed amongst multiple systems. The computing environment 100 may also be specifically dedicated to the implementation of the present technology. As a person in the art of the present technology may appreciate, multiple variations as to how the computing environment 100 is implemented may be envisioned without departing from the scope of the present technology.

Those skilled in the art will appreciate that processor 110 is generally representative of a processing capability. In some embodiments, in place of or in addition to one or more conventional Central Processing Units (CPUs), one or more specialized processing cores may be provided. For example, one or more Graphic Processing Units 111 (GPUs), Tensor Processing Units (TPUs), and/or other so-called accelerated processors (or processing accelerators) may be provided in addition to or in place of one or more CPUs.

System memory will typically include random access memory 130, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 120 is shown as an example of a mass storage device, but more generally such mass storage may comprise any type of non-transitory storage device configured to store data, programs, and other information, and to make the data, programs, and other information accessible via a system bus 160. For example, mass storage may comprise one or more of a solid state drive, hard disk drive, a magnetic disk drive, and/or an optical disk drive.

Communication between the various components of the computing environment 100 may be enabled by a system bus 160 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wired or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the computing device 100 in addition to or instead of the touchscreen 190.

According to some implementations of the present technology, the solid-state drive 120 stores program instructions suitable for being loaded into the random access memory 130 and executed by the processor 110 for executing acts of one or more methods described herein. For example, at least some of the program instructions may be part of a library or an application.

### Retinal Signal Data Processing System

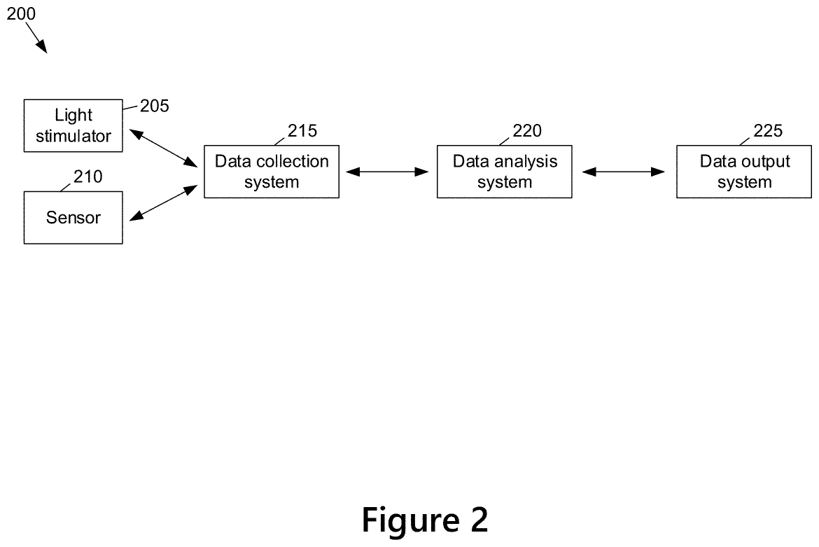

FIG. 2 is a block diagram of a retinal signal data processing system 200 in accordance with various embodiments of the present technology. The retinal signal data processing system 200 may collect retinal signal data from an individual. As described above, when compared with conventional ERG, the retinal signal data captured using the retinal signal data processing system 200 may comprise additional features and/or data, such as impedance, a higher measurement frequency, an extended range of retinal light stimulation, and/or a longer measurement time. The retinal signal data processing system 200 may process and/or analyse the collected data. The retinal signal data processing system 200 may output retinal signal data after detecting and/or removing artifacts from the retinal signal data, such as distortions or interferences.

It is to be expressly understood that the system 200 as depicted is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The retinal signal data processing system 200 may comprise a light stimulator 205, which may be an optical stimulator, for providing light stimulation signals to the retina of an individual. The retinal signal data processing system 200 may comprise a sensor 210 for collecting electrical signals that occur in response to the optical stimulation. The retinal signal data processing system 200 may comprise a data collection system 215, which may be a computing environment 100, for controlling the light stimulator 205 and/or collecting data measured by the sensor 210. For example the light stimulator 205 and/or sensor 210 may be a commercially available ERG system such as the Espion Visual Electrophysiology System from DIAGNOSYS, LLC or the UTAS and RETEVAL systems manufactured by LKC TECHNOLOGIES, INC.

The light stimulator 205 may be any kind of light source or sources which, alone or in combination, can generate light within a specified range of wavelength, intensity, frequency and/or duration. The light stimulator 205 may direct the generated light onto the retina of an individual. The light stimulator 205 may comprise light-emitting diodes (LEDs) in combination with other light sources, such as one or more Xenon lamps. The light stimulator 205 may provide a background light source.

The light stimulator 205 may be configured to provide a light stimulation signal to the retina of an individual. The retinal signal data collected may depend upon the light stimulation conditions. In order to maximise the potential to generate relevant retinal signal features in the retinal signal data, the light stimulator 205 may be configured to provide a large variety of light conditions. The light stimulator 205 may be configurable to control the background light and/or the stimulation light directed onto the retina as light flashes.

The light stimulator 205 may comprise any sources of light able to generate light beams of different wavelength (e.g. from about 300 to about 800 nanometers), light intensity (e.g. from about 0.001 to about 3000 cd·s/m²), illumination time (e.g. from about 1 to about 500 milliseconds), time between each light flashes (e.g. about 0.2 to about 50 seconds) with different background light wavelength (e.g. from about 300 to about 800 nanometers) and background light intensity (e.g. about 0.01 to about 900 cd/m²).

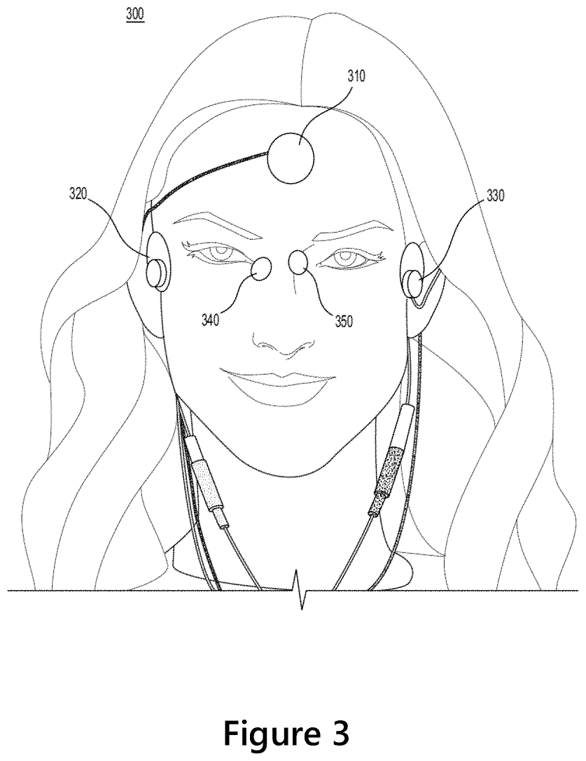

The retinal signal data processing system 200 may comprise a sensor 210. The sensor 210 may be arranged to detect electrical signals from the retina. The sensor 210 may comprise one or more electrodes. The sensor 210 may be an electroretinography sensor. FIG. 3, described below, illustrates an example of electrode placement. A ground electrode may be placed on the skin in the middle of the forehead. Reference electrodes for each eye may be placed on the earlobes, temporal areas near the eyes, forehead, and/or other skin areas. The ground electrode may serve as the zero reference for the positive or negative polarity of the electrical signals. The ground electrode may be located at the center of the forehead, on top of the head, and/or on the wrist. Any part of the circuit involved in collecting the electrical signals may benefit from real-time impedance monitoring.

Electrical signals from the retina may be triggered by light stimulation from the light stimulator 205 and collected by the sensor 210 as retinal signal data. The retinal signal data may be collected by the sensor 210 such as by an electrode positioned on the ocular globe or nearby ocular areas. The light may trigger an electrical signal of low amplitude generated by the retinal cells of the individual. Depending upon the nature of the light (e.g. intensity, wavelength, spectrum, frequency and duration of the flashes) and the conditions for the light stimulation (e.g. background light, dark or light adaptation of the individual subjected to this process), different electrical signals may be generated because different types of retinal cells will be triggered. This signal propagates within the eye and ultimately to the brain visual areas via the optic nerve. However, as any electrical signal, it propagates in all possible directions depending upon the conductivity of the tissues. Therefore the electrical signal may be collected in the tissues external to the ocular globe, accessible from outside, such as the conjunctiva.

There are several types of electrodes which can be used to collect the electrical signals; they are based upon specific material, conductivity, and/or geometry. It should be understood that there are many possible designs of recording electrodes and that any suitable design or combination of designs may be used for the sensor 210. The sensor 210 may comprise e.g., contact lens, foil, wire, corneal wick, wire loops, microfibers, and/or skin electrodes. Each electrode type has its own recording characteristics and inherent artifacts.

The electrical signals originating from the retina in response to a light stimulus are collected by means of a circuit formed with different electrodes, such as electrodes of the sensor 210. The circuit may include pre-amplifiers, amplifiers, filters, analog-to-digital converters, and/or any other electrical signal processing devices. The electrical signals may be collected as a potential difference between an electrode (called ‘active’ electrode) placed in the region where the electrical signal is received from the retina (e.g. the cornea or the ocular globe) and an electrode placed nearby that location (called ‘reference’ electrode). The electric potential difference is often collected relative to an electrical neutral point relative to a ground electrode.

In addition to the sensor 210, the system 200 may also include other devices to monitor and record light stimulation wavelength and/or light intensity. These devices may include a spectrometer, a photometer, and/or any other devices for collecting light characteristics. The light stimulation wavelength and/or light intensity may have an impact on the quantity of light stimulation reaching the retina and therefore triggering the retinal signal in response to this stimulus. The collected light stimulation wavelength and/or light intensity data may be included in the retinal signal data. The collected light stimulation wavelength and/or light intensity data may be used to adjust various values of the retinal signal data. These adjustments may be performed after collection of the retinal signal data and/or in real-time during collection of the retinal signal data.

In addition to the sensor 210, the system 200 may also include other devices to monitor eye position and/or pupil size (e.g. a camera to track pupil positioning and aperture), both having an impact on the quantity of stimulation light reaching the retina and therefore affecting the electrical signals triggered in response to this stimulus. The eye position and/or pupil size data may be included in the retinal signal data. This data may be used in order to adjust the retinal signal data during and/or after collection of the retinal signal data.

The electrical signals may be obtained between the active electrode (positioned onto the eye or near the eye) and the reference electrode. The electrical signals may be obtained with or without differential recording from the ground electrode. The electrodes of the sensor 210 may be connected to a data collection system 215, which may comprise a recording device. Prior to being recorded, the electrical signals may pass through any number of pre-amplifiers, amplifiers, filters, analog-to-digital converters, and/or any other signal processing devices. The data collection system 215 may allow for amplification of the electrical signals and/or conversion of the electrical signals to digital signal for further processing. The data collection system 215 may implement frequency filtering processes that may be applied to the electrical signals from the sensor 210. The data collection system 215 may store data describing the electrical signals in a database, such as in the format of voltage versus time points.

The data collection system 215 may be arranged to receive measured electrical signals of an individual, such as from the sensor 210, and/or stimulating light data, such as from the light stimulator 205, and store this collected data as retinal signal data. The data collection system 215 may be operatively coupled to the light stimulator 205 which may be arranged to trigger the electrical signals and provide the data to the data collection system 215. The data collection system 215 may synchronize the light stimulation with the electrical signal capture and recording. The data collection system 215 may capture calibration data prior to a flash of light and retinal signal data after the flash of light. The calibration data and the retinal signal data may have the same parameters and use the same circuit.

The collected data may be provided to the data collection system 215 via any suitable method, such as via a storage device (not shown) and/or a network. The data collection system 215 may be connectable to the sensor 210 and/or the light stimulator 205 via a communication network (not depicted). The communication network may be the Internet and/or an Intranet. Multiple embodiments of the communication network may be envisioned and will become apparent to the person skilled in the art of the present technology.

The retinal signal data may comprise electrical response data (e.g. voltage and circuit impedance) collected for several signal collection times (e.g. 5 to 500 milliseconds) at several sampling frequencies (e.g. 0.2 to 24 kHz) with the light stimulation synchronization time (time of flash) and/or offset (baseline voltage and impedance prior to light stimulation). The data collection system 215 may collect retinal signal data at frequencies (i.e. sampling rate) of 4 to 16 kHz, or higher. This frequency may be higher than conventional ERG. The electrical response data may be collected continuously or intermittently.

The retinal signal data may comprise impedance measurements and/or other electrical parameters. The retinal signal data may comprise optical parameters such as pupil size changes, retinal area illuminated, and/or applied luminance parameters (intensity, frequency of light, frequency of signal sampling). The retinal signal data may comprise population parameters such as age, gender, iris pigmentation, retinal pigmentation, and/or skin pigmentation as a proxy for retinal pigmentation, etc. The retinal signal data may comprise admittance, conductance, and/or susceptance data.

The data collection system 215 may comprise a sensor processor for measuring the impedance of the electrical circuit used to collect the retinal signal data. The impedance of the electrical circuit may be recorded simultaneously with the capture of other electrical signals. The collected impedance data may be stored in the retinal signal data. The method to determine the impedance of the circuit simultaneously with the capture of the electrical signals may be based upon a process of injecting a reference signal of known frequency and amplitude through the recording channel of the electrical signals. This reference signal may then be filtered out separately and processed. By measuring the magnitude of the output at the excitation signal frequency, the electrode impedance may be calculated. Impedance may then be used as a co-variable to enhance signal density with the resistance of the circuit at each time point of the recording of the electrical signals.

The data analysis system 220 may process the retinal signal data collected by the data collection system 215. The data analysis system 220 may use recorded signal data and/or other information (related to the process for collecting the retinal signal data) to build the retinal signal data and/or to remove artifactual components from the retinal signal data. The data collection system 215 may implement any of the methods 800, 900, and/or 1000 (described in further detail below) for processing the retinal signal data. The data analysis system 220 may extract retinal signal features and/or descriptors from the retinal signal data, and/or perform any other processing on the retinal signal data.

The data output system 225 may output data collected by the data collection system 215. The data output system 225 may output results generated by the data analysis system 220. The data output system 225 may output predictions, such as the predicted likelihood that an individual is subject to one or more conditions, such as a mental condition. For each condition, the output may indicate the predicted likelihood that the individual is subject to that condition. The output may be used by a clinician to aid in determining whether an individual is subject to a medical condition and/or determining which medical condition the individual is subject to.

The data collection system 215, data analysis system 220, and/or data output system 225 may be accessed by one or more users, such as through their respective clinics and/or through a server (not depicted). The data collection system 215, data analysis system 220 and/or data output system 225 may also be connected to retinal signal data management software which could further extract retinal signal features and analyse embedded biosignatures and/or biomarkers. The data collection system 215, data analysis system 220, and/or data output system 225 may be connected to appointment management software which could schedule appointments or follow-ups based on the determination of the condition by embodiments of the system 200.

The data collection system 215, data analysis system 220, and/or data output system 225 may be distributed amongst multiple systems and/or combined within a system or multiple systems. The data collection system 215, data analysis system 220, and/or data output system 225 may be geographically distributed.

FIG. 3 is a diagram 300 of exemplary electrode placement for collecting retinal signal data in accordance with various embodiments of the present technology. A ground electrode 310 may be placed on the skin in the middle of the forehead. The ground electrode 310 may serve as the zero reference for the positive or negative polarity of the electrical signals collected by reference electrodes 320, 330, 340, and 350. The reference electrodes 320, 330, 340, and 350 capture electrical signals emitted from the individual. A circuit may be formed using the ground electrode 310 and/or reference electrodes 320, 330, 340, and 350. Various parameters of the circuit may be recorded, such as the current, voltage, impedance, and/or any other electrical parameters. The ground electrode 310 and reference electrodes 320, 330, 340, and 350 may be any type of electrode, may have any shape, may be made of any suitable material, and/or may be any combination of different types of electrodes. For example the ground electrode 310 may be a first type of electrode and the reference electrodes 320, 330, 340, and 350 may be a second type of electrode that is different from the first type of electrode.

It should be understood that the diagram 300 is an example of one arrangement of electrodes on an individual, and that any number of electrodes may be used and/or the electrodes may be placed in any other suitable areas. For example the ground electrode 310 may be placed on the individual's wrist instead of the forehead.

Movement of the ground electrode 310 and/or reference electrodes 320, 330, 340, and/or 350 during data collection may cause artifacts in the retinal signal data. The methods described below may be used to alert the clinician that artifacts are occurring, compensate for artifacts in the retinal signal data, and/or re-record retinal signal data that has been affected by artifacts. These methods may reduce and/or remove the effects of electrodes placed in positions that may cause artifacts to occur in the retinal signal data. By using the methods described below, any errors that occur when placing the electrodes and/or collecting the data may be compensated for and/or the effects of those errors may be reduced.

### Method for Removing Distorted Signals

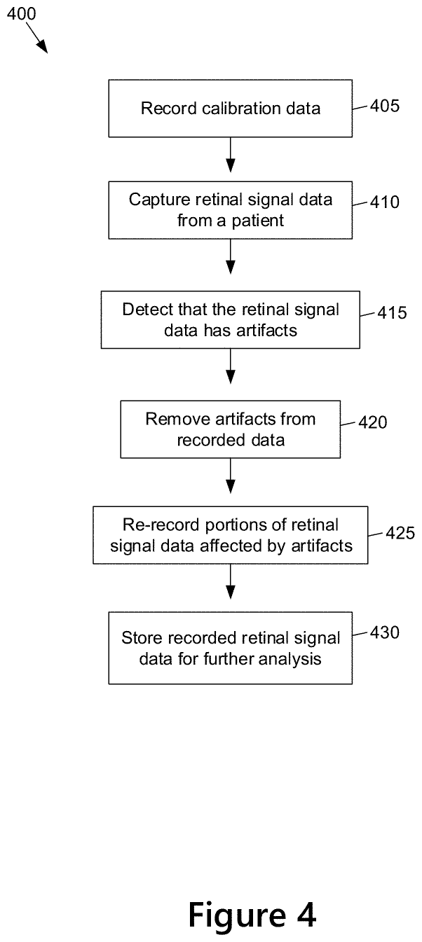

FIG. 4 is a flow diagram of a method 400 for compensating for artifacts in retinal signal data in accordance with various embodiments of the present technology. The retinal signal data may be or have been recorded using the retinal signal data processing system 200. All or portions of the method 400 may be executed by the data collection system 215, data analysis system 220, and/or the prediction output system 225. In one or more aspects, the method 400 or one or more steps thereof may be performed by a computing system, such as the computing environment 100. The method 400 or one or more steps thereof may be embodied in computer-executable instructions that are stored in a computer-readable medium, such as a non-transitory mass storage device, loaded into memory and executed by a CPU. The method 400 is exemplary, and it should be understood that some steps or portions of steps in the flow diagram may be omitted and/or changed in order.

At step 405, calibration data may be collected. The calibration data may be collected during a pre-determined time period, such as 20 milliseconds. During the collection of the calibration data, the retina of the individual might not be stimulated by the optical stimulators. In other words, the individual might not be exposed to any light stimulation during the recording of the calibration data. Electrical parameters and/or any other data may be collected at step 405. The current, voltage, impedance, and/or any other electrical parameters may be collected.

Baseline parameters may be determined at step 405, such as a baseline current, voltage, impedance, and/or any other parameters. The baseline parameters may be determined based on the calibration data. The baseline parameters may be a mean/and or a median of the parameters recorded in the calibration data. For example a baseline impedance may be determined as a mean of the impedance recorded in the calibration data. The baseline parameters may be used for all later measurements. For example an average voltage may be determined, and this average voltage may be subtracted from later measurements, such as those performed at step 410.

At step 410, retinal signal data may be captured from an individual. The retinal signal data may include co-variables and parameters which may impact on the nature and the quality of the retinal signal data, such as the parameters of light stimulation and the impedance of the receiving electrical circuit used to collect the retinal signal data. The electrical circuit may be implemented in a device. The retinal signal data may include measured electrical signals captured by electrodes placed on the individual. The retinal signal data may include parameters of the system used to capture the retinal signal data, such as the parameters of light stimulation. The retinal signal data may include the impedance of the receiving electrical circuit measuring the electrical signals.

The retinal signal data may comprise impedance measurements and/or other electrical parameters. The retinal signal data may comprise parameters such as eye position, pupil size, intensity of applied luminance, frequency of light stimulation, frequency of retinal signal sampling, wavelength of illumination, illumination time, background light wavelength, and/or background light intensity. The retinal signal data may comprise clinical information cofactors such as age, gender, iris pigmentation, retinal pigmentation, and/or skin pigmentation as a proxy for retinal pigmentation, etc. Therefore, in certain embodiments, the method 400 comprises at step 410, collecting impedance measurements. The same set of parameters may be recorded at steps 405 and 410.

To generate the retinal signal data, the retina of an individual may be stimulated, such as by using the light stimulator 205 which may be one or more optical stimulators. The retinal signal data may be collected by a sensor, such as the sensor 210, which may comprise one or more electrodes and/or other sensors.

The light stimulator may comprise any sources of light able to generate light beams of different wavelength (e.g. from about 300 to about 800 nanometers), light intensity (e.g. from about 0.01 to about 3000 cd·s/m2), illumination time (e.g. from about 1 to about 500 milliseconds), time between each light flashes (e.g. about 0.2 to about 50 seconds) with different background light wavelength (e.g. from about 300 to about 800 nanometers) and background light intensity (e.g. about 0.01 to about 900 cd/m2).

The retinal signal data may comprise electrical response data (e.g. voltage and circuit impedance) collected for several signal collection times (e.g. 5 to 500 milliseconds) at several sampling frequencies (e.g. 0.2 to 24 kHz) with the light stimulation synchronisation time (time of flash) and offset (baseline voltage and impedance prior to light stimulation). Therefore, step 410 may comprise collecting retinal signal data at frequencies of 4 to 16 kHz.

The baseline parameters may also be used as the offset for the current, voltage, and/or any other electrical parameters. For example, the voltage and/or current may be normalized based on the baseline voltage and/or baseline current.

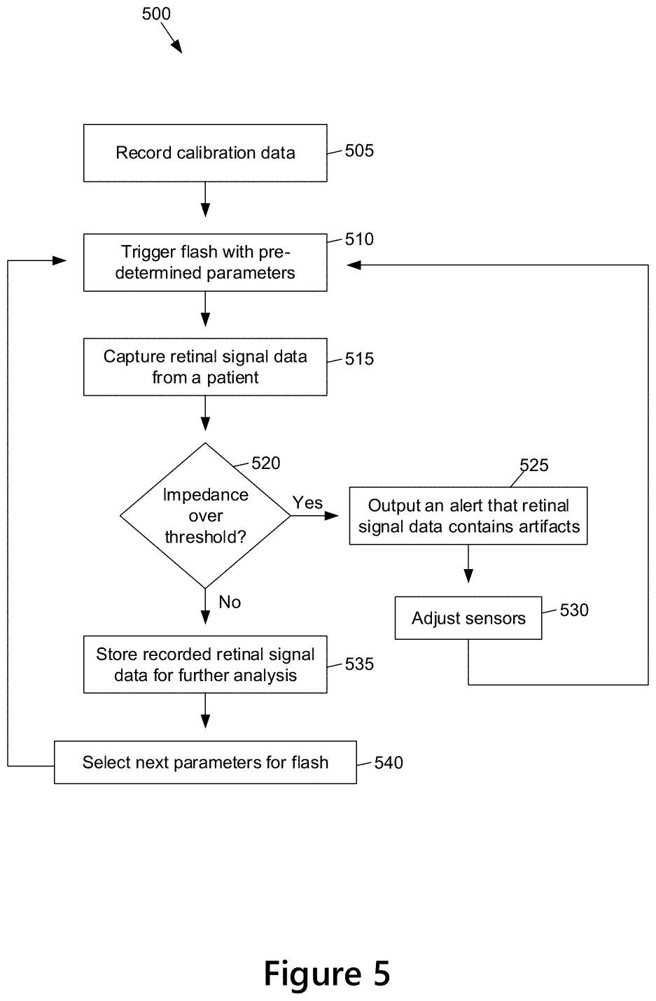

Steps 405 and 410 may be repeated to collect the retinal signal data. Prior to each flash from the light stimulator 205, the calibration data may be recorded at step 405. For example the calibration data may be collected for 20 ms prior to the flash, and then retinal signal data may be collected after the flash at step 410. Then, prior to the next flash, calibration data may be recorded at step 405. FIG. 5 describes this sequence in more detail.

After the retinal signal data is collected, such as by a practitioner, the retinal signal data may be uploaded to a server, such as the data analysis system 220, for analysis. The retinal signal data may be stored in a memory 130 of the computer system. In other embodiments, the retinal signal data is uploaded to the data analysis system 220 in real-time, while the retinal signal data is being collected.

At step 415, the collected retinal signal data may be determined to have artifacts, such as distorted signals, in the data. Distorted signals may include spikes or other unusual features. Artifacts in electrical signals recorded from any electrode placed on the tissues of an individual may have a direct impact on amplitude, impedance, admittance, and/or conductance (the ability for electrical charges to flow in a certain path) of the circuit that the electrode is part of. These artifacts may be detected by analysing the time-course of the retinal signal data and locating the changes in amplitude, impedance, admittance, and/or conductance that may indicate artifacts. The retinal signal data may be determined to be likely to contain artifacts based an amount of change of the impedance of the circuit and/or a rate of change of the impedance.

The retinal signal data may be compared to pre-determined criteria or patterns to determine whether artifacts exist in the retinal signal data. For example, sudden changes in slope and/or baseline and/or high variations in amplitude and/or impedance in a very short period of time may be identified as indicative of artifacts. The rate of change of parameters of the retinal signal data may be analyzed to determine whether artifacts are present, such as the rate of change of impedance. The artifacts may be in the recorded electrical signals of the retinal signal data and/or any other type of data contained within the retinal signal data.

The impedance in the collected retinal signal data may be compared to the baseline impedance determined using the calibration data recorded at step 405. A threshold impedance may be determined based on the calibration data. For example the threshold impedance may be ten percent higher than the baseline impedance determined at step 405. If the impedance of the retinal signal data is above the threshold at any time, the retinal signal data may be determined to contain artifacts. A time period corresponding to the impedance being above the threshold may be determined. The retinal signal data recorded during that time period may be labeled as containing artifacts and/or the retinal signal data corresponding to that time period may be deleted.

At step 420 the artifacts may be removed from the retinal signal data. The dynamic characteristics of the circuit used to collect the electrical signals may be used to determine which parts of the retinal signal data contain artifacts. For example, changes in conductance in the circuit including the ‘active’ electrode and the ‘reference’ electrode, or in the circuit with the electrical neutral point relative to a ground electrode, are parameters used to detect and remove artifacts. The lower the impedance of the circuits used to collect the electrical signals, the better the quality of the collected electrical signals. The impedance of suitable circuits to collect retinal signal data is typically below 5 kohms. In some cases, an impedance as low as 100 ohms for the circuit including the ‘active’ electrode and the ‘reference’ electrode may be achieved with appropriate electrodes and circuit.

Artifacts may be detected, compensated for, and/or removed, using real time impedance measurements to rectify the collected electrical signals with regard to the conductivity of the circuit collecting the signals. The electrical signals may be adjusted based on characteristics of the stimulus (e.g., light intensity, light spectrum, retinal surface illuminated) that triggered the electrical signals. These adjustments may remove and/or compensate for artifacts, such as by adjusting the amplitude of the current and/or voltage.

Time periods corresponding to the artifacts may be determined, and all or a portion of the signals recorded during those time periods may be rectified or removed. The artifacts may be removed from the retinal signal data and/or ignored for subsequent signal analysis. For example time periods in the retinal signal data may be labeled as corresponding to artifacts. The data collected during those time periods might not be used later when the retinal signal data is being analysed.

Working at a higher sampling frequency and/or collecting a higher volume of signal information may minimize the impact of removing any artifacts. To a certain extent, signals may also be corrected by considering the dynamics of the receiving circuit, which is based upon adding conductance to the features of the retinal signal data (as an additional retinal signal feature for the retinal signal data).

The retinal signal data responsive to an individual flash may be determined to contain artifacts, and all retinal signal data responsive to that flash might be removed from the retinal signal data. A subset of the retinal signal data responsive the flash might be removed. For example the electrical signals may be recorded for 200 ms, and the impedance of the recording circuit might be below the threshold impedance for the first 150 ms, and then above the threshold impedance for the last 50 ms. The retinal signal data for the first 150 ms might be stored and used for further processing, whereas the retinal signal data for the last 50 ms might not be stored and used for further processing.

At step 425, retinal signal data may be re-recorded. Portions of the retinal signal data may be determined to be likely to have artifacts. These time periods may be determined based on the impedance being above a threshold during the recording of the retinal signal data. Instead of or in addition to removing the artifacts at step 420, the portions of the retinal signal data that have been affected by artifacts may be re-recorded. The stimulus that was applied to the individual during the time periods when artifacts were detected may be re-applied, and the electrical signals produced in response to that stimulus may be recorded. The impedance may be monitored during the capture of the electrical signals. If the impedance remains below the threshold impedance, which indicates that the re-recorded data likely does not contain artifacts, the re-recorded data may be stored as retinal signal data. The original portions of the retinal signal data that contained artifacts may be replaced by the re-recorded data.

At step 430, the recorded retinal signal data may be stored for further analysis. The retinal signal data may be used for predicting whether an individual is subject to a condition, such as a mental disorder. Although the method 400 is described herein as being applied to retinal signal data, it should be understood that the method 400 may be applied to any other type of collected signal data.

### Method for Providing Distorted Signal Alert

FIG. 5 is a flow diagram of a method 500 for detecting artifacts and outputting an alert during collection of retinal signal data in accordance with various embodiments of the present technology. All or portions of the method 500 may be executed by the data collection system 215, data analysis system 220, and/or the prediction output system 225. In one or more aspects, the method 500 or one or more steps thereof may be performed by a computing system, such as the computing environment 100. The method 500 or one or more steps thereof may be embodied in computer-executable instructions that are stored in a computer-readable medium, such as a non-transitory mass storage device, loaded into memory and executed by a CPU. The method 500 is exemplary, and it should be understood that some steps or portions of steps in the flow diagram may be omitted and/or changed in order.

At step 505, calibration data may be recorded. Actions performed at step 505 may be similar to those described above with regard to step 405 of the method 400. Baseline and/or threshold parameters may be determined based on the calibration data. For example, a baseline and threshold impedance may be determined at step 505.

At step 510, a flash of light may be triggered with pre-determined parameters. The parameters of the flash of light may include a luminance, a wavelength, an illumination time, a background light wavelength, and/or a background light intensity.

At step 515, retinal signal data may be captured from an individual. Actions performed at step 510 may be similar to those described above with regard to step 410 of the method 400. An indicator of the parameters of the flash of light triggered at step 510 may be stored with the corresponding retinal signal data captured at step 515.

At step 520, the collected retinal signal data may be compared to the threshold impedance determined at step 505 based on the calibration data. The retinal signal data may be determined to contain artifacts and/or be likely to contain artifacts if the retinal signal data collected at step 515 was above the threshold at any time. The impedance of the circuit collecting the retinal signal data may be compared to the threshold impedance. If the impedance of the circuit collecting the retinal signal data was above the threshold at any time, the retinal signal data may be determined to contain artifacts. Actions performed at step 515 may be similar to those described above with regard to step 415 of the method 400. Although step 520 describes comparing the impedance to the threshold impedance, any other indicator of the circuit's dynamic resistance may be used. For example a threshold admittance and/or a threshold susceptance may be determined. The admittance and/or susceptance of the circuit collecting the retinal signal data collected at step 515 may be compared to the threshold admittance and/or threshold susceptance. If the admittance and/or susceptance is above the threshold at any time, then the collected retinal signal data may be determined to contain artifacts at step 520.

The artifact detection may be performed while the retinal signal data is being collected, such as in real-time or near real-time. The retinal signal data may be continuously monitored and/or monitored at pre-determined time periods. All or a portion of the retinal signal data may be monitored to determine whether there are any artifacts in the data. The artifacts may appear in the data regarding electrical signals in the retinal signal data, such as the amplitude of the current and/or voltage of the collected electrical signals.

The retinal signal data may be compared to pre-determined criteria or patterns to determine whether artifacts exist in the retinal signal data. For example, sudden changes in slope and/or baseline and/or high variations in amplitude and/or impedance in a very short period of time may be identified as indicative of artifacts. The artifacts may be in the recorded electrical signals of the retinal signal data and/or any other type of data contained within the retinal signal data.

If the impedance surpasses the threshold impedance and/or artifacts are detected using any other technique, the method 500 may continue at step 525. At step 525, an alert may be output that artifacts have been detected. The alert may be issued after one or more artifacts have been detected in the retinal signal data. The alert may be issued when the impedance is above the threshold impedance. For example an alert may be output if an electrode were to change location or move during the recording. Any drift due to e.g. eye movement or eye blinks may cause an alert to be output. The alert may be issued after artifacts have been detected for a threshold time period, such as two seconds. The alert may indicate which sensor is causing the artifacts. An alert may be output based on a sudden change in slope and/or baseline and/or high variations in amplitude and/or impedance. The alert may be an audio alert and/or a visual alert.

At step 530 an operator may adjust the data collection system 215, sensor 210, and/or light stimulator 205 based on the alert. The operator may adjust one or more sensors and/or any other part of the data collection system. The operator may be notified whether the adjustment succeeded in correcting the issue, such as by the notification being cleared. Steps 525 and 530 are optional.

After step 530, the flash of light may be triggered again at step 510 with the same parameters. The corresponding retinal signal data may be captured at step 515 and at step 520 the retinal signal data may be compared to the threshold impedance to determine whether the retinal signal data contains artifacts. If the retinal signal data does not surpass the threshold impedance, the method 500 may continue to step 535. Otherwise, if the retinal signal data again has artifacts, then the method 500 may proceed to step 525 and the same flash of light may be triggered at step 510.

At step 535, the retinal signal data may be stored. The retinal signal data may be stored for further analysis, such as for predicting whether the individual is subject to a medical condition. The retinal signal data may be stored with the parameters of the flash that was triggered at step 510. Actions performed at step 535 may be similar to those described above with regard to step 430 of the method 400. Although the method 500 is described herein as being applied to retinal signal data, it should be understood that the method 500 may be applied to any retinal signal data and/or any other type of collected signal data.

At step 540, a next set of parameters may be selected for the flash. A sequence of flash parameters may have been pre-determined, and the next set of parameters may be selected from the pre-determined sequence. If there are no more parameters to select, the method 500 may end. Otherwise, the method 500 may continue to step 510 and the flash may be triggered with the selected parameters.

Rather than checking the impedance at step 520 after each flash is triggered, the artifact detection may be performed after all flashes have been triggered or after a series of flashes have been triggered. For example a series of flashes for a first luminance may be triggered, and retinal signal data may be captured for each flash, and then the impedance of the retinal signal data may be compared to the threshold impedance for each flash to determine whether any of the retinal signal data may contain artifacts. Then, a series of flashes for a second luminance may be triggered. Prior to each flash, the calibration data may be collected and a threshold impedance may be determined for each individual flash.

### Method for Removing Distorted Signals Using an MLA

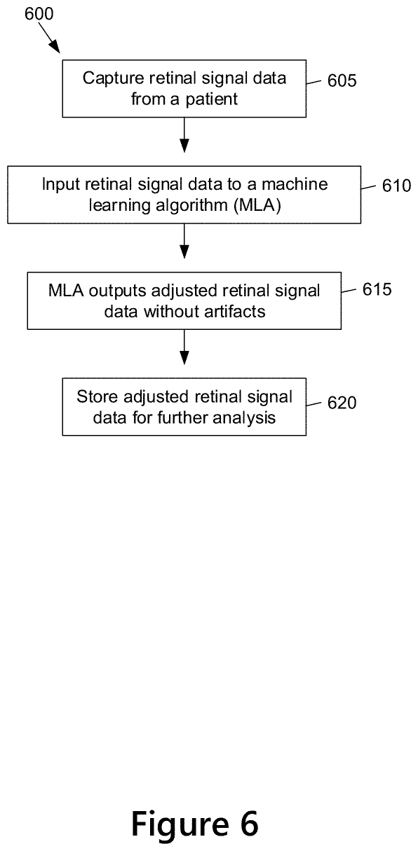

FIG. 6 is a flow diagram of a method 600 for using a machine learning algorithm (MLA) to remove artifacts from retinal signal data in accordance with various embodiments of the present technology. All or portions of the method 600 may be executed by the data collection system 215, data analysis system 220, and/or the prediction output system 225. In one or more aspects, the method 600 or one or more steps thereof may be performed by a computing system, such as the computing environment 100. The method 600 or one or more steps thereof may be embodied in computer-executable instructions that are stored in a computer-readable medium, such as a non-transitory mass storage device, loaded into memory and executed by a CPU. The method 600 is exemplary, and it should be understood that some steps or portions of steps in the flow diagram may be omitted and/or changed in order.

At step 605, retinal signal data may be captured from an individual. The retinal signal data may be retinal signal data. Actions performed at step 605 may be similar to those described above with regard to step 410 of the method 400. Calibration data may be captured as well, such as prior to triggering the retinal signal data.

At step 610, all or a portion of the captured retinal signal data may be input to a machine learning algorithm (MLA). Calibration data may also be input to the MLA. The MLA may identify portions of the retinal signal data that contain artifacts. The MLA may be based on any suitable MLA architecture, such as a neural network, and may include one or more MLAs.

The MLA may remove artifacts based upon predefined thresholds in the dynamics of the receiving circuit, e.g. threshold in impedance or signal amplitude, or baseline, or changes of those parameters. The MLA may also remove artifacts based upon learned patterns obtained from signals with known artifacts, discriminate between various types of artifacts such as signal distortions, and/or removed unwanted signals not generated from the retina. Each of these individual tasks may be performed by a separate MLA. The MLA may output a reconstructed signal without the artifacts.

The MLA may be trained based on labeled training data. The labeled training data may include datasets of retinal signal data that is impacted by artifacts with known origins. The label may indicate the nature of the artifacts (e.g., electrodes displacement, blinks, ocular movements, and/or signal distortions such as drifts or interferences). After being trained, the MLA may be able to predict time periods in which artifacts occur. The MLA may also predict a cause of the artifacts.

The MLA may be used to make predictions based on previously recorded data and/or data being recorded in real-time. If the MLA is used during signal collection, the MLA may output a notification when artifacts are detected.

At step 615, the MLA may output adjusted retinal signal data with artifacts having been removed. The artifacts may be compensated for, such as by replacing the artifacts with other data, or rectifying the distorted signal, or ignoring the part of the signal where artifacts have been detected.

At step 620, the adjusted retinal signal data may be stored. Actions performed at step 620 may be similar to those described above with regard to step 430 of the method 400. Although the method 600 is described herein as being applied to retinal signal data, it should be understood that the method 600 may be applied to any retinal signal data and/or any other type of collected signal data.

### Method for Predicting Likelihood of Medical Condition

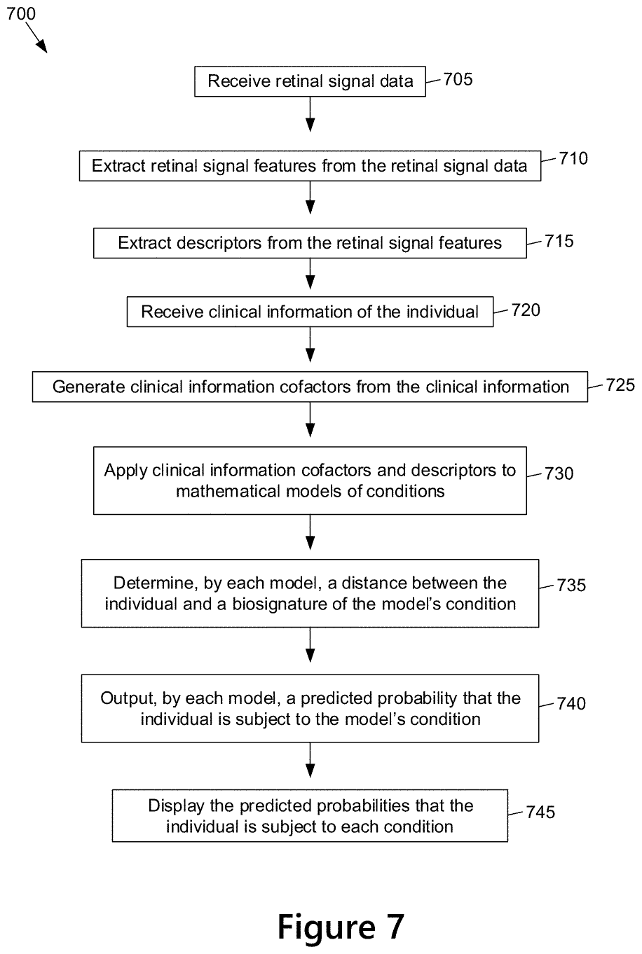

FIG. 7 is a flow diagram of a method 700 for predicting a likelihood of a medical condition in accordance with various embodiments of the present technology. All or portions of the method 700 may be executed by the data collection system 215, data analysis system 220, and/or the prediction output system 225. In one or more aspects, the method 700 or one or more steps thereof may be performed by a computing system, such as the computing environment 100. The method 700 or one or more steps thereof may be embodied in computer-executable instructions that are stored in a computer-readable medium, such as a non-transitory mass storage device, loaded into memory and executed by a CPU. The method 700 is exemplary, and it should be understood that some steps or portions of steps in the flow diagram may be omitted and/or changed in order.

The method 700 comprises performing various activities such as extracting retinal signal features from retinal signal data, selecting the most relevant retinal signal features to specific conditions, combining and comparing those retinal features to generate mathematical descriptors most discriminant to the conditions to be analysed or compared, generating multimodal mapping, identifying biomarkers and/or biosignatures of the conditions, and/or predicting a likelihood that a patient us subject to any one of the conditions, as will now be described in further detail below.

At step 705, retinal signal data may be received. The retinal signal data may have been captured using a pre-defined collection protocol. The retinal signal data may include measured electrical signals captured by electrodes placed on the patient. The retinal signal data may include parameters of the system used to capture the retinal signal data, such as the parameters of light stimulation. The retinal signal data may include the impedance of the receiving electrical circuit used in the device measuring the electrical signals.

The retinal signal data may comprise impedance measurements and/or other electrical parameters. The retinal signal data may comprise optical parameters such as pupil size changes, and/or applied luminance parameters (intensity, wavelength, spectrum, frequency of light stimulation, frequency of retinal signal sampling).

After the retinal signal data is collected, such as by a practitioner, the retinal signal data may be uploaded to a server, such as the data analysis system 220, for analysis. The retinal signal data may be retrieved from the data analysis system 220 at step 705. The retinal signal data may be stored in a memory 130 of the computer system.

The retinal signal data received at step 705 may have been collected and/or processed to reduce, remove, and/or compensate for artifacts, such as using any one of the methods 400, 500, and/or 600. As discussed above, portions of the retinal signal data may be flagged as containing artifacts, such as portions of the circuit collecting the retinal signal data that surpassed a threshold impedance. The flagged data might not be used for the following steps of the method 700. For example, if the retinal signal data corresponding to an individual flash were determined to have artifacts, the retinal signal data corresponding to that flash might not be used in the following steps of the method 700.

At step 710, retinal signal features may be extracted from the retinal signal data. The extraction of retinal signal features may be based upon the processing of the retinal signal data and/or their transforms using multiple signal analysis methods, such as polynomial regressions, wavelet transforms, and/or empirical mode decomposition (EMD). The extraction of retinal signal features may be based upon parameters derived from those analyses or specific modeling, e.g. principal components and most discriminant predictors, parameters from linear or non-linear regression functions, frequency of higher magnitude, Kullback-Leibler coefficient of difference, features of the gaussian kernels, log likelihood of difference and/or areas of high energy. These analyses may be used to determine the contribution of each specific retinal signal feature and compare the retinal signal features statistically.

The retinal signal features to be extracted may have been previously determined. The retinal signal features to extract may have been determined by analyzing labeled datasets of retinal signal data for multiple patients. Each patient represented in the datasets may have one or more associated medical conditions that the patient is subject to and/or one or more medical conditions that the patient is not subject to. These medical conditions may be the label to each patient's dataset. By analyzing a set of retinal signal data from patients sharing a medical condition, the retinal signal features to extract may be determined. A multi-modal map may be generated based on the retinal signal features. Domains may be determined based on the multi-modal map.

At step 715, descriptors may be extracted from the retinal signal features. The mathematical descriptors may be mathematical functions combining features from the retinal signal data and/or clinical cofactors. The descriptors may indicate a retinal signal feature specific to a condition or a population in view of further discrimination between groups of patients. The descriptors may be selected to obtain the components of a biosignature which together contributes the most to mathematical models of the conditions, by e.g. match-merging descriptors and cofactors using mathematical expressions or relations, by using e.g. PCA, SPCA or other methods used in selecting and/or combining retinal signal data features.

At step 720, clinical information of the individual may be received. The clinical information may include medical records and/or any other data collected regarding the individual. The clinical data may include the results of a questionnaire and/or clinical examination by a healthcare practitioner.

At step 725, clinical information cofactors may be generated using the clinical information. The clinical information cofactors may be selected based on their influence on the retinal signal data. The clinical information cofactors may include indications of the individual's age, gender, skin pigmentation which may be used as a proxy for retinal pigmentation, and/or any other clinical information corresponding to the individual.

At step 730, the clinical information cofactors and/or the descriptors may be applied to mathematical models of conditions. Any number of mathematical models may be used. A clinician may select which mathematical models to use. Each model may correspond to a specific condition or a control.

At step 735, each model may determine a distance between the patient and the biosignature of the model's condition. Main components of the retinal signal data may be located within domains corresponding to the conditions. The descriptors and/or clinical information cofactors may be compared to each model's biosignature.

At step 740, each model may output a predicted probability that the individual is subject to the model's condition. The likelihood that the individual is subject to a condition may be predicted based upon the level of statistical significance in comparing the magnitude and the location of the descriptors of the individual to those in the model. The predicted probability may be binary and indicate that the biosignature of the condition is either present or absent in the individual's retinal signal data. The predicted probability may be a percentage indicating how likely it is that the individual is subject to the condition.

At step 745, the predicted probability that the individual is subject to each condition may be output. An interface and/or report may be output. The interface may be output on a display. The interface and/or report may be output to a clinician. The output may indicate a likelihood that the individual is subject to one or more conditions. The output may indicate a positioning of the individual within a pathology. The predicted probabilities may be stored.

The output may include determining a medical condition, the predicted probability of a medical condition, and/or a degree to which retinal signal data of the individual is consistent with the condition and/or other conditions. The predicted probability may be in the format of a percentage of correspondence for the medical condition, which may provide an objective neurophysiological measure in order to further assist in a clinician's medical condition hypothesis.

The output may be used in conjunction with a clinician's provisional medical condition hypothesis to increase the level of comfort with the clinician's determination of a medical condition and/or start an earlier or more effective treatment plan. The output may be used to begin treatment earlier rather than spending additional time clarifying the medical condition and the treatment plan. The output may reduce the clinician's and/or individual's level of uncertainty of the clinician's provisional medical condition hypothesis. The output may be used to select a medication to administer to the individual. The selected medication may then be administered to the individual.

The method 700 may be used to monitor a condition of an individual. An individual may have been previously diagnosed with a condition. The method 700 may be used to monitor the progress of the condition. The method 700 may be used to monitor and/or alter a treatment plan for the condition. For example the method 700 may be used to monitor the effectiveness of a medication being used to treat the condition. The retinal signal data may be collected before, during, and/or after the individual is undergoing treatment for the condition.

The method 700 may be used to identify and/or monitor neurological symptoms of an infection, such as a viral infection. For example the method 700 may be used to identify and/or monitor neurological symptoms of individuals who were infected with COVID-19. Retinal signal data may be collected from individuals that are or were infected with COVID-19. The retinal signal data may be assessed using the method 700 to determine whether the patient is suffering from neurological symptoms, a severity of the neurological symptoms, and/or to develop a treatment plan for the neurological symptoms.

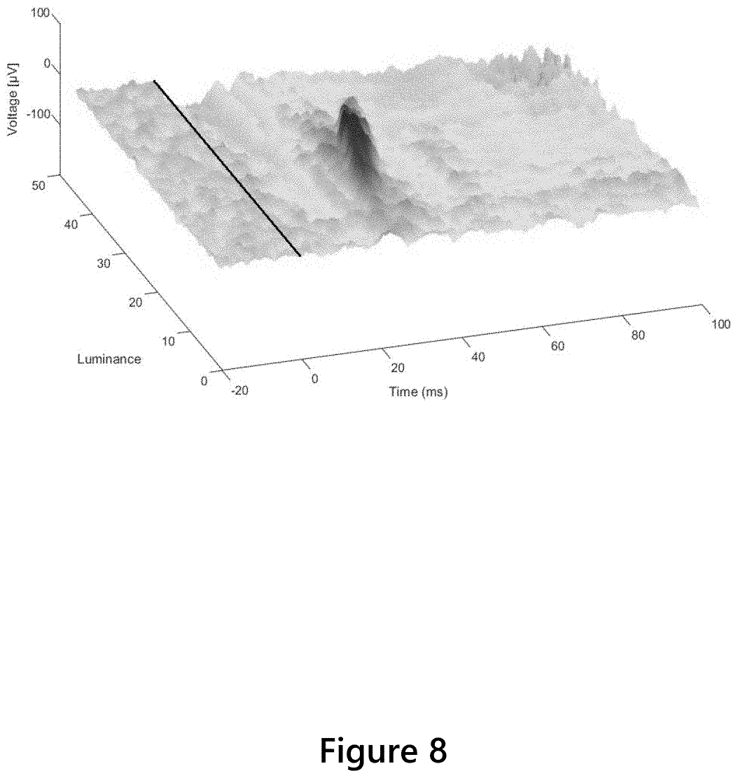

FIG. 8 is a three-dimensional retinal signal data generated with 45 incremental light intensities (luminance steps) from 0.4 cd·sec/m² to 794 cd·sec/m² in photopic conditions (accommodation to background light) with a sampling frequency of 16 kHz in accordance with various embodiments of the present technology. The recording starts 20 milliseconds prior to triggering the retinal signals (light stimulation at 0 millisecond indicated by the black line) in order to determine the baseline amplitude values for each light stimulation luminance.

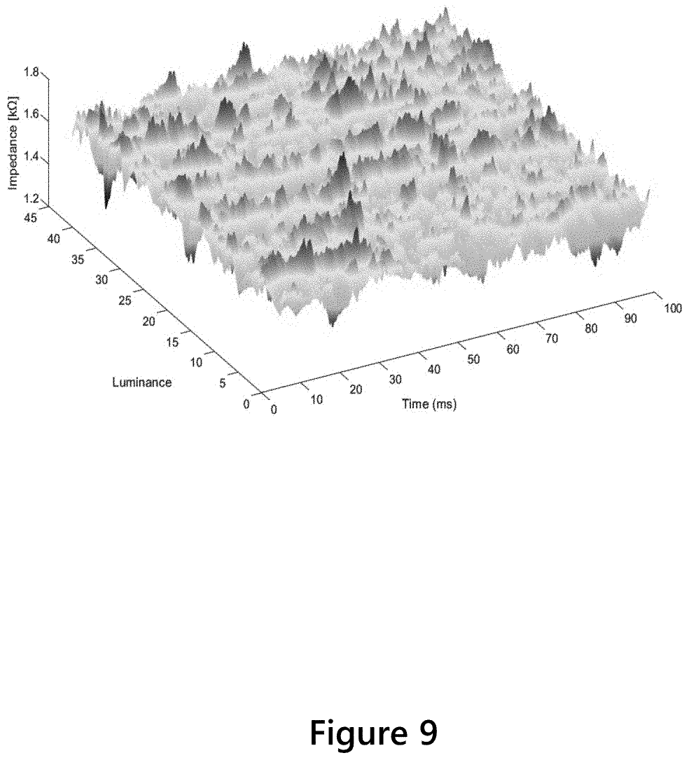

FIG. 9 is a three-dimensional impedance of retinal signal data generated with 45 incremental light intensities (luminance) from 0.4 cd·sec/m² to 794 cd·sec/m² in photopic conditions (accommodation to background light) and impedance capture simultaneously with the amplitude of the retinal signal at a sampling frequency of 16 kHz in accordance with various embodiments of the present technology. The retinal signal is triggered at 0 millisecond for each 45 light intensities.

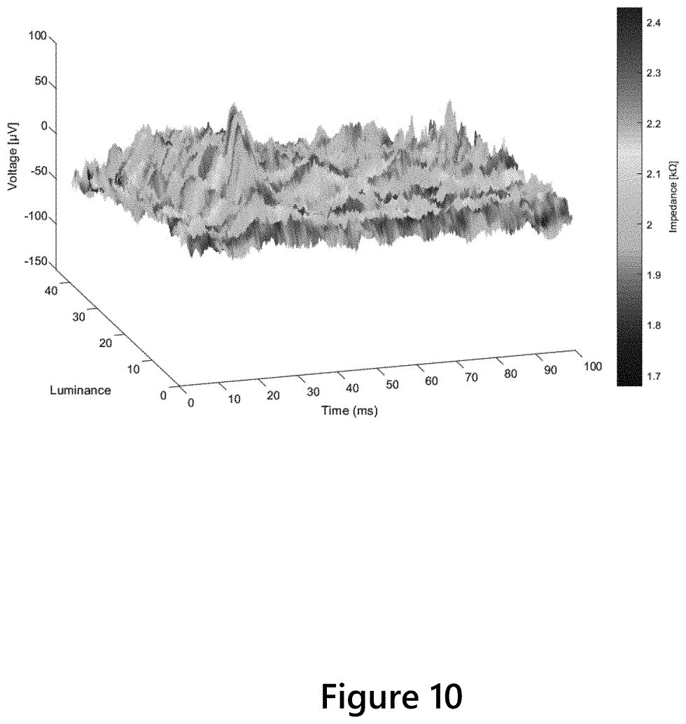

FIG. 10 is a four-dimensional retinal signal data (amplitude vs impedance vs stimulation light luminance vs time) generated with 45 incremental light intensities (luminance) from 0.4 cd·sec/m² to 794 cd·sec/m² in photopic conditions (accommodation to background light) and simultaneous impedance capture with a sampling frequency of 16 kHz in accordance with various embodiments of the present technology. Colors indicate the impedance values as per the color scale at the right of the Figure. Baseline impedance are generally lower than 2 kohms, and do not significantly vary during the retinal signal recording, except in case of artifacts, electrode displacement or signal interference.

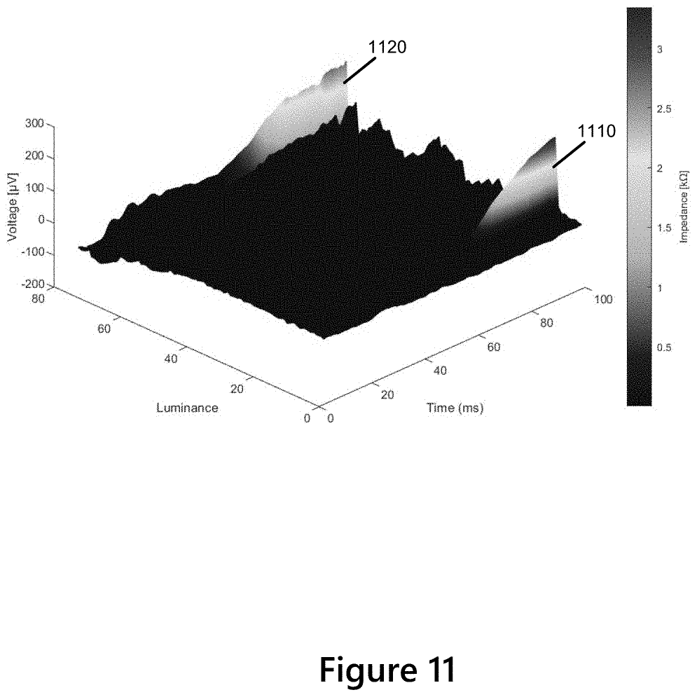

FIG. 11 is a four-dimensional retinal signal data (amplitude vs impedance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz in accordance with various embodiments of the present technology. Colors indicate the impedance values as per the color scale at the right of the Figure. Changes in impedance are found during the signal recording at luminance 9 (0.9 cd·sec/m²) and 72 (624 cd·sec/m²), with impedance higher than baseline values not exceeding 500 ohms, which indicates two distortions are present in the signal. The artifact 1110 at luminance 9 may have been caused by electrode displacement and/or loss of contact. The artifact 1120 at luminance 72 may have been caused by signal drift.

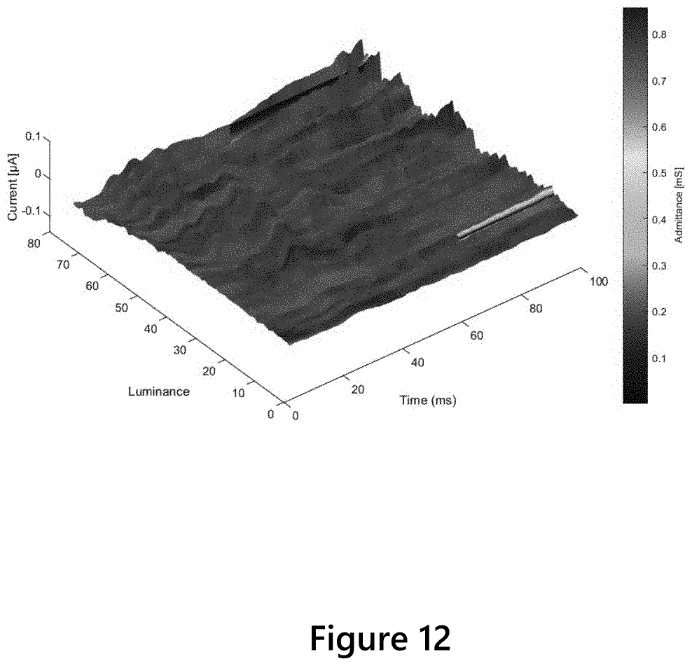

FIG. 12 is a four-dimensional retinal signal (current vs admittance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz in accordance with various embodiments of the present technology. Colors indicate the admittance values as per the color scale at the right of the Figure. The changes in impedance found during the signal recording presented in FIG. 11, respectively at luminance 9 (0.9 cd·sec/m²) and 72 (624 cd·sec/m²) have been rejected by the present technology and the signal has been corrected accordingly as shown by the values of amplitudes and admittance.

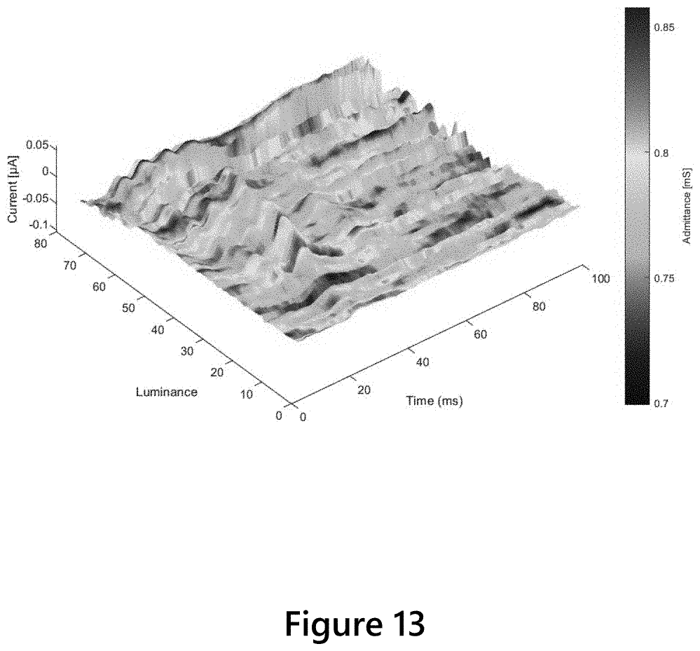

FIG. 13 is a four-dimensional retinal signal (current vs admittance vs stimulation light luminance vs time) generated with 75 incremental light intensities (luminances) from 0.4 cd·sec/m² to 851 cd·sec/m² in photopic conditions (accommodation to background light) with a sampling frequency of 4 kHz. Colors indicate the admittance values as per the color scale at the right of the Figure. The two distortions found in the retinal signal presented in FIG. 11, respectively at luminance 9 (0.9 cd·sec/m²) and 72 (624 cd·sec/m²), have been corrected.

The techniques, systems, and methods described herein may be applied to any type of signals where electrode positioning and conductance is directly related to the quality of the recorded signals, i.e. allow removing components which are not related to the signal itself, or adjusting for e.g. electrodes displacement.

1. A method executed by at least one processor of a computing system, the method comprising:
receiving retinal signal data corresponding to an individual;
determining that there are one or more artifacts in the retinal signal data by determining that an impedance of a circuit that collected the retinal signal data has surpassed a threshold impedance of the circuit;
modifying the retinal signal data to compensate for the artifacts; and
storing the retinal signal data.

2. The method of claim 1, wherein modifying the retinal signal data to compensate for the artifacts comprises removing at least a portion of the retinal signal data corresponding to the artifacts.

3. The method of claim 1, further comprising:
receiving calibration data corresponding to the individual; and
determining, based on the calibration data, the threshold impedance of the circuit.

4. The method of claim 1, wherein the retinal signal data is responsive to at least one flash of light from a light stimulator, wherein the calibration data is collected prior to the at least one flash of light by the same circuit that collected the retinal signal data, and wherein the method further comprises causing the light stimulator to generate the at least one flash of light.

5. The method of claim 1, wherein the retinal signal data has a sampling frequency between 4 to 24 kHz, and wherein the retinal signal data is collected for a signal collection time of 200 milliseconds to 500 milliseconds.

6. (canceled)

7. The method of claim 1, wherein the one or more artifacts comprise distortions in the retinal signal data.

8. The method of claim 1, wherein the one or more artifacts were caused by one or more of: capture of electrical signals not originating from the retina, shift in electrode positioning, change in ground or reference electrode contact, photomyoclonic reflex, eye lid blinks, and ocular movements.

9. The method of claim 1, further comprising:
extracting, from the retinal signal data, one or more retinal signal features;
extracting, from the retinal signal features, one or more descriptors;
applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and
outputting the first predicted probability and the second predicted probability.

10. A method executed by at least one processor of a computing system, the method comprising:
receiving retinal signal data corresponding to an individual;
determining that there are one or more artifacts in the retinal signal data by determining that an impedance of a circuit that collected the retinal signal data has surpassed a threshold impedance of the circuit;
storing an indication in the retinal signal data of time periods corresponding to the one or more artifacts; and
storing the retinal signal data.

11. The method of claim 10, further comprising:
receiving calibration data corresponding to the individual; and
determining, based on the calibration data, the threshold impedance of the circuit.

12. The method of claim 10, further comprising determining the time periods corresponding to the one or more artifacts by determining the time periods that an impedance of the retinal signal data surpasses the threshold impedance.

13. The method of claim 10, wherein the retinal signal data is responsive to at least one flash of light from a light stimulator, wherein the calibration data is collected prior to the at least one flash of light, and wherein the method further comprises causing the light stimulator to generate the at least one flash of light.

14. The method of claim 10, wherein the retinal signal data has a sampling frequency between 4 to 24 kHz, and wherein the retinal signal data is collected for a signal collection time of 200 milliseconds to 500 milliseconds.

15. (canceled)

16. The method of claim 10, wherein the one or more artifacts comprise distortions in the retinal signal data.

17. The method of claim 10, wherein the one or more artifacts were caused by one or more of: capture of electrical signals not originating from the retina, shift in electrode positioning, change in ground or reference electrode contact, photomyoclonic reflex, eye lid blinks, and ocular movements.

18. The method of claim 10, further comprising:
extracting, from the retinal signal data, one or more retinal signal features;
extracting, from the retinal signal features, one or more descriptors;
applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and
outputting the first predicted probability and the second predicted probability.

19. A method executed by at least one processor of a computing system, the method comprising:
recording a first set of retinal signal data corresponding to an individual;
determining that there are one or more artifacts in the first set of retinal signal data by determining that an impedance of a circuit that collected the first set of retinal signal data has surpassed a first threshold impedance of the circuit;
recording a second set of retinal signal data corresponding to the individual;
determining that the impedance of the circuit while recording the second set of retinal signal data has not surpassed a second threshold impedance of the circuit; and
storing the second set of retinal signal data.

20. The method of claim 19, further comprising:
recording a first set of calibration data corresponding to the individual before recording the first set of retinal signal data;
determining, based on the first set of calibration data, the first threshold impedance of the circuit;
recording a second set of calibration data corresponding to the individual before recording the second set of retinal signal data; and
determining, based on the second set of calibration data, the second threshold impedance of the circuit.

21. The method of claim 20, further comprising:
after recording the first set of calibration data, triggering a light stimulator to generate a first flash of light based on a set of flash parameters, wherein the first set of retinal signal data is responsive to the first flash of light; and
after recording the second set of calibration data, triggering the light stimulator to generate a second flash of light based on the set of flash parameters, wherein the second set of retinal signal data is responsive to the second flash of light.

22-23. (canceled)

24. The method of claim 19, further comprising:
extracting, from the second set of retinal signal data, one or more retinal signal features;
extracting, from the retinal signal features, one or more descriptors;
applying the one or more descriptors to a first mathematical model and a second mathematical model, wherein the first mathematical model corresponds to a first condition and the second mathematical model corresponds to a second condition, thereby generating a first predicted probability for the first condition and a second predicted probability for the second condition; and
outputting the first predicted probability and the second predicted probability.

25-33. (canceled)
